(19) United States
(12) Patent Application Publication
(10) Pub. No.: US 2021/0128527 A1
(43) Pub. Date: May 6, 2021

(54) COMPOSITIONS AND METHODS FOR TREATING HEMORRHAGIC STROKE

(71) Applicants: Graham Timmins, Albuquerque, NM (US); Ke Jian Liu, Albuquerque, NM (US); Rong Pan, Albuquerque, NM (US); UNM Rainforest Innovations, Albuquerque, NM (US)

(72) Inventors: Graham Timmins, Albuquerque, NM (US); Ke Jian Liu, Albuquerque, NM (US); Rong Pan, Albuquerque, NM (US)

(21) Appl. No.: 16/770,438

(22) PCT Filed: Dec. 7, 2018
(86) PCT No.: PCT/US2018/064456

§ 371 Date: Jun. 5, 2020

Related U.S. Application Data
(60) Provisional application No. 62/596,158, filed on Dec. 8, 2017.

(51) Int. Cl. A61K 31/409; A61K 31/4439; A61K 31/47; A61K 31/4709; A61K 31/517; A61K 31/5377; A61K 31/4985; A61K 31/506; A61K 31/4706; A61K 31/496; A61K 31/437; A61P 9/10

(52) U.S. Cl. CPC A61K 31/409; A61K 31/4439; A61K 31/47; A61K 31/4709; A61K 31/517; A61P 9/10; A61K 31/4985; A61K 31/506; A61K 31/4706; A61K 31/496; A61K 31/437; A61K 31/5377

(57) ABSTRACT

A pharmaceutical composition includes a ferrochelatase inhibitor and a pharmaceutically acceptable carrier. In another aspect, a method of treating a subject having, or a t risk of having, a hemorrhagic stroke generally includes administering to the subject a pharmaceutical composition that includes a ferrochelatase inhibitor in an amount effective to ameliorate at least one symptom or clinical sign of hemorrhagic stroke.

### CROSS-REFERENCE TO RELATED APPLICATION

This application claims priority to U.S. Provisional Patent Application No. 62/596,158, filed Dec. 8, 2017, which is incorporated herein by reference in its entirety.

### SUMMARY

This disclosure describes, in one aspect, a pharmaceutical composition that includes a ferrochelatase inhibitor and a pharmaceutically acceptable carrier.

In some embodiments, the ferrochelatase inhibitor can be an isomer of a protoporphyrin compound or a chemically modified protoporphyrin analog. In some of these embodiments, the protoporphyrin analog can include a reduction of at least one protoporphyrin vinyl group or an N-alkylation. In some of these embodiments, the N-alkylation can include an N-methylation such as, for example, N-methyl protoporphyrin IX.

In some embodiments, the ferrochelatase inhibitor can include a protein kinase inhibitor. In some of these embodiments, the protein kinase inhibitor can include axitinib, cabozantinib, crenolanib, erlotinib, gefitinib, linsitinib, momelotinib, neratinib, nilotinib, pelitinib, vargatef, vemurafenib, AEW-541, ARRY-380, AZD-2014, AZD-5438, AZD-8055, BGT-226, BMS-690514, CP-724714, CUDC-101, Cyc-116, E-7080, GSK-1070916, GSK-690693, MK-2461, MK-8033, OSI-027, or R-406.

In another aspect, this disclosure describes a method of treating a subject having, or at risk of having, a hemorrhagic stroke. Generally, the method includes administering to the subject any embodiment of the pharmaceutical composition summarized above in an amount effective to ameliorate at least one symptom or clinical sign of hemorrhagic stroke.

In some embodiments, the pharmaceutical composition is administered to the subject before the subject manifests a symptom or clinical sign of hemorrhagic stroke. In other embodiments, the pharmaceutical composition is administered to the subject after the subject manifests a symptom or clinical sign of hemorrhagic stroke.

In some embodiments, the amount of the pharmaceutical composition effective to ameliorate at least one symptom or clinical sign of hemorrhagic stroke is an amount effective to inhibit protoporphyrin IX from combining with Zn to form zinc protoporphyrin (ZnPP).

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Hemorrhage leads to zinc accumulation, which was associated with cell death. Labile zinc or cell death on the brain slices was detected by fluorescence probe Fluozin-3. Relative Fluozin-3 fluorescence intensity indicates the Fluozin-3 fluorescence intensity in each brain slice normalized to the average value of saline group (C: contralateral hemisphere; H: hemorrhagic hemisphere). Bar graph data are presented as mean±SEM, n=3. *P<0.05.

FIG. 2. Brain injury was showed by T2-weighted magnetic resonance imaging. The brightness of T2-weighted images indicated the severity of brain damage. White line shows the brain damage in hemorrhagic hemisphere. Arrow indicates hyperintense lesion in the white matter of contralateral hemisphere. The relative brain damage on the y-axis of the bar graph means the percentage of brain damage volume in each whole brain normalized to the average percentage of saline group. Scale bar: 1 mm. Data are presented as mean±SEM, n=5. *P<0.05.

FIG. 3. ZnPP generated following hemorrhagic stroke. ZnPP on the brain slices was imaged by collecting its autofluorescence. Scale bar: 1 mm. Relative ZnPP fluorescence intensity is defined as the ZnPP fluorescence intensity in each brain slice normalized to the average value of saline group.

FIG. 4. ZnPP fluorescence spectrum in each hemisphere of rats was measured by fluorescence spectrophotometer. Sham: hemisphere tissue collected from sham (saline-injected) rats; Hem: hemisphere tissue collected from ICH rats; Hem TPEN: TPEN-pretreated ICH rats; C: contralateral hemisphere; H: hemorrhagic hemisphere. “Subtracted fluorescence intensity” for an experimental group was calculated by subtracting the fluorescent intensity observed in the contralateral hemisphere of sham rats from the fluorescence intensity observed in the experimental group. Quantification of ZnPP in each hemorrhage was calculated according to ZnPP standard curve of the fluorescence intensity at 588 nm (n=4). Data are presented as mean±SEM. *P<0.05, versus Sham H, #P<0.05, versus Hem H.

FIG. 5. SFC-MS scanning showed mass spectroscopic profile of commercial pure ZnPP, collagenase-induced hemorrhagic and saline injected control brain (n=3). Peak identity was assigned by retention time and chromatographic pattern of authentic standard.

FIG. 6. Hemorrhage caused decreased oxygenation in tissue surrounding hematoma area. LiPc crystal (arrow) was implanted in brain before collagenase injection (C: contralateral hemisphere; H: hemorrhagic hemisphere). Tissue oxygen level before (Pre-hemo) and 24 hours after (Post-hemo) collagenase injection was measured by in vivo electron paramagnetic resonance oximetry (n=3). Data are presented as mean±SEM. #P<0.05, versus pre-hemo group.

FIG. 7. Hypoxia, zinc, and blood are three factors in ZnPP generation following ICH. (A) ZnPP fluorescence spectrum in each hemisphere of rats was measured by fluorescence spectrophotometer. Sham: hemisphere tissue collected from sham rats; MCAO: hemisphere tissue collected from MCAO rats; C: contralateral hemisphere; I: Ischemic hemisphere. “Subtracted fluorescence intensity” for an experimental group was calculated by subtracting the fluorescent intensity observed in the contralateral hemisphere of sham rats from the fluorescence intensity observed in the experimental group. Quantification of ZnPP in blood was calculated according to ZnPP standard curve of the fluorescence intensity at 588 nm (n=4). Data are presented as mean±SEM. *P<0.05, versus Normoxia control, #P<0.05, versus Hypoxia Zn. (B) Hemorrhage was visualized by NaBH₄ staining. (C) ZnPP fluorescence spectrum in each blood was measured by fluorescence spectrophotometer. Quantification of ZnPP in blood was calculated according to ZnPP standard curve of the fluorescence intensity at 588 nm (n=3). Data are presented as mean±SEM. *P<0.05, versus normoxia, #P<0.05, versus hypoxia plus zinc. (D) ZnPP fluorescence spectrum in each blood was measured by fluorescence spectrophotometer, with and without TPEN pretreatment. Quantification of ZnPP in blood was calculated according to ZnPP standard curve of the fluorescence intensity at 588 nm (n=3). Data are presented as mean±SEM. *P<0.05, versus normoxia, #P<0.05, versus hypoxia plus zinc.

FIG. 8. ZnPP is toxic to hypoxic brain cells and tissue. Primary neurons or astrocytes were incubated with indicated concentration (μM) of ZnPP or Hemin. After a three-hour exposure to normoxia or hypoxia, cell death was measured by Cytotox 96 nonradioactive cytotoxicity assay kit. Data are presented as mean±SEM, n=3. *P<0.05, versus same concentration ZnPP in normoxia group, #P<0.05, versus same concentration Hemin both in hypoxia group.

FIG. 9. Brain infarct was shown by T2-weighted MRI. (C: contralateral hemisphere; I: ischemic hemisphere). White line indicates the hyperintense lesion. Scale bar: 1 mm. The percentage of brain infarct volume showed the hyperintense lesion area in the whole brain slice. Data are presented as mean±SEM, n=3. *P<0.05, versus saline group. The subtracted infarct volume bar graph shows the percentage of hyperintense lesion volume in the whole brain with ZnPP injection subtracting the average percentage of hyperintense lesion volume in the whole brain with saline injection in indicated condition. Data are presented as mean±SEM, n=3. *P<0.05.

FIG. 10. ZnPP is generated by ferrochelatase catalysis; inhibition of ferrochelatase decreased ZnPP generation and toxicity. (A) ZnPP in the hemorrhagic brain was detected by imaging its autofluorescence on brain slices. Scale bar for the whole brain is 1 mm. Scale bar for the enlarged region is 100 μm. Relative ZnPP fluorescence intensity means the ZnPP fluorescence intensity in each brain slice normalized to the average value of saline group. Data are presented as mean±SEM, n=3. *P<0.05. (B) Cell death in the hemorrhagic brain was measured by TUNEL assay kit (C: contralateral hemisphere; H: hemorrhagic hemisphere). Arrow showed the samples of TUNEL positive cells. Scale bar for the whole brain is 1 mm. Scale bar for the enlarged region is 100 μm. Data are presented as mean±SEM, n=3. *P<0.05. (C) Brain damage was showed by T2-weighted MRI. Scale bar: 1 mm. The relative brain damage means the percentage of damage volume in each whole brain normalized to the average percentage of saline group. Data are presented as mean±SEM, n=5. *P<0.05.

FIG. 11. Schematic representation of the heme degradation pathway. HO-1 degrades heme and produces CO, biliverdin and Fe′. Biliverdin is converted to bilirubin while Fe′ generation increases in ferritin. CO, bilirubin and ferritin are neuroprotective factors. Our finding (yellow circle) shown that endogenous ZnPP generation may cause brain injury by inhibition the activity of HO-1.

### DETAILED DESCRIPTION OF ILLUSTRATIVE EMBODIMENTS

This disclosure describes compositions and methods related to treating hemorrhagic stroke.

Hemorrhagic stroke responsible for about 40 percent of all stroke death. The lysis of red blood cells, hemin release, and overload of iron are recognized as causes of intracerebral hemorrhage (ICH)-induced brain damage. This disclosure reports a finding that zinc is also involved in the ICH-induced brain damage. Hemorrhagic stroke caused a significant accumulation of zinc around the hemorrhagic area, where massive cell death occurred. Pre-treatment of rats with a zinc-specific chelator greatly decreased zinc accumulation and brain damage following ICH. In the absence of a zinc-specific chelator, zinc protoporphyrin (ZnPP) was formed and accompanied zinc accumulation during ICH. ZnPP is toxic to brain cells under hypoxic conditions but not under normoxic conditions. ICH-induced hypoxia, zinc accumulation, and blood release are three factors in ZnPP generation following ICH. Finally, the inhibition of ferrochelatase remarkably reversed the hemorrhagic-induced ZnPP generation and brain damage. It indicates that ZnPP was formed by ferrochelatase catalyzing the insertion of zinc into free protoporphyrin, which contributed in brain damage following ICH. The results suggest that zinc mediates brain damage via ZnPP generation after ICH and identifies compounds that inhibit the formation of ZnPP can have therapeutic utility in treating hemorrhagic stroke.

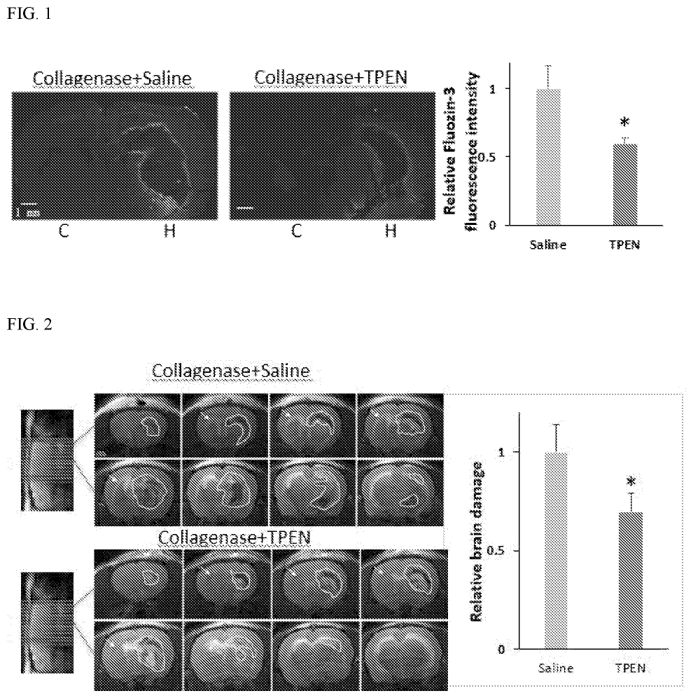

Collagenase injection was used to induce intracerebral hemorrhage and labile zinc accumulated on the edge of hemorrhagic area after hemorrhagic stroke. Selective zinc-specific fluorescence probe Fluozin-3 was used to probe the labile zinc accumulation in brain tissue after hemorrhagic stroke. As shown in FIG. 1, there was little detectable signal in the non-hemorrhagic hemisphere while strong fluorescence signal was observable around hemorrhagic area. By pretreating the rats with zinc-specific chelator TPEN one hour before collagenase injection, the zinc fluorescence signal was significantly reduced.

Zinc accumulation is associated with cell death after hemorrhagic stroke. To evaluate the effect of ICH-induced zinc accumulation on brain damage, the cell death in brain slices was measured by TUNEL assay. Abundant TUNEL-positive cells were observed at the edge of hemorrhagic area. Chelating zinc with TPEN, however, markedly decreased the TUNEL signal. The result suggests that hemorrhagic-stroke-induced labile zinc accumulation promoted cell death. Moreover, by using T2-weighted magnetic resonance imaging (MRI) to evaluate the brain damage, the T2-WI showed an area of signal loss and a hyperintense lesion on the hemorrhagic hemisphere and white matter on the contralateral hemisphere 24 hours after collagen-induced ICH (FIG. 2). The hyperintense lesion was significantly reduced by chelating labile zinc by TPEN pretreatment, indicating that elevated levels of labile zinc are involved in in hemorrhagic stroke-induced brain damage.

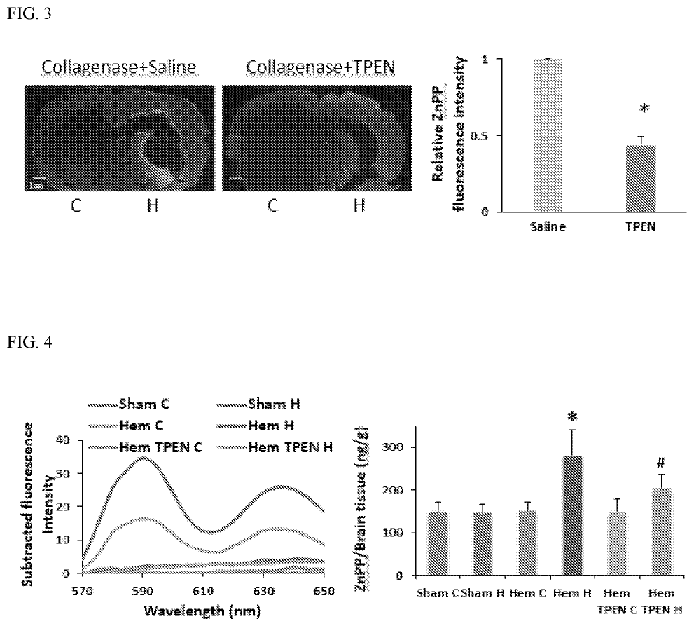

Zinc accumulates at the brain parenchyma following ICH. Zinc protoporphyrin (ZnPP) levels were measured in each hemisphere at 24 hours after collagenase injection. Since ZnPP is fluorescent with a strong fluorescence peak at 588 nm and a weak fluorescent peak at around 630 nm (excitation wavelength: 420), ZnPP generation was imaged in the hemorrhagic rat brain by fluorescence microscope. As shown in FIG. 3, there was little ZnPP in the non-hemorrhagic hemisphere, while ZnPP generation was visible in the hemorrhagic hemisphere. The ZnPP generated around the hemorrhagic area (shown in FIG. 3) strongly co-localized with increased levels of free zinc (shown in FIG. 1). The tissue slices shown in FIG. 3 are the same as the tissue slices shown in FIG. 1). To confirm that ZnPP formation was dependent on increased free zinc, TPEN was used to chelate zinc. ZnPP fluorescence was significantly decreased by TPEN (P=0.0034, FIG. 3), demonstrating that ZnPP generation is dependent upon increased free zinc. To confirm that the fluorescence signal is from ZnPP rather than some other fluorescent product, brain tissue was collected and subjected to fluorescence spectrophotometry to detect the fluorescence spectrum. The fluorescence intensity at 588 nm was increased at the hemorrhagic hemisphere and TPEN treatment significantly reduced the degree to which ZnPP formed at the hemorrhagic hemisphere increase (FIG. 4).

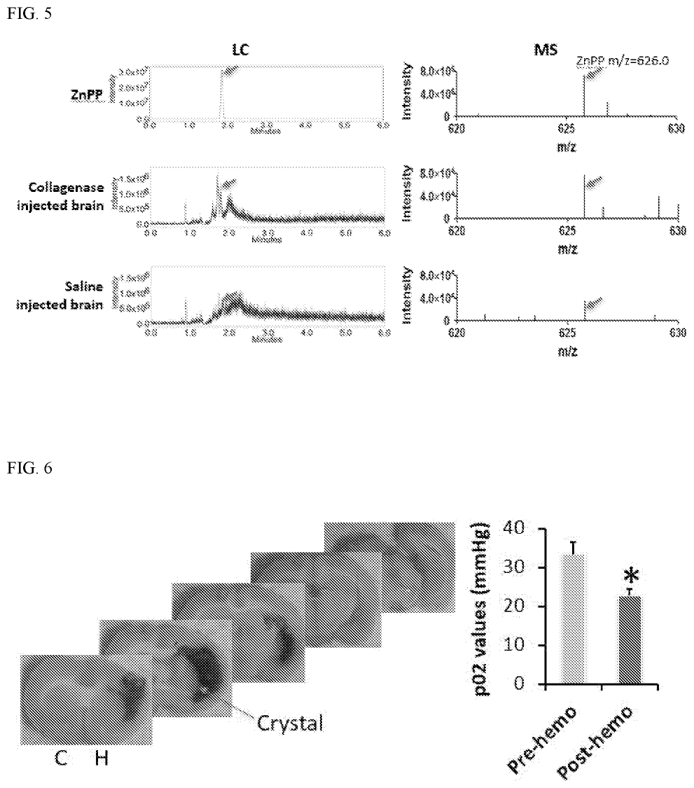

The presence of ZnPP in hemorrhagic and normal brain was authenticated by SFC-MS spectroscopy. FIG. 5 shows mass spectroscopic profile of standard ZnPP (ZnPP), hemorrhagic brain (Collagenase injected), and normal brain (Saline injected). The standard ZnPP give a peak at 626 m/z value. The ZnPP peak is also found in both hemorrhagic and normal brains, however, the intensity of the peak was much higher in hemorrhagic brain compared to normal brain. Peak identity was assigned by retention time and chromatographic pattern of authentic standard. The LCMS results suggest that hemorrhagic stroke leads to ZnPP formation, while the decrease of ZnPP level by TPEN pre-treatment suggests that the ZnPP generation is caused by zinc accumulation following hemorrhagic stroke.

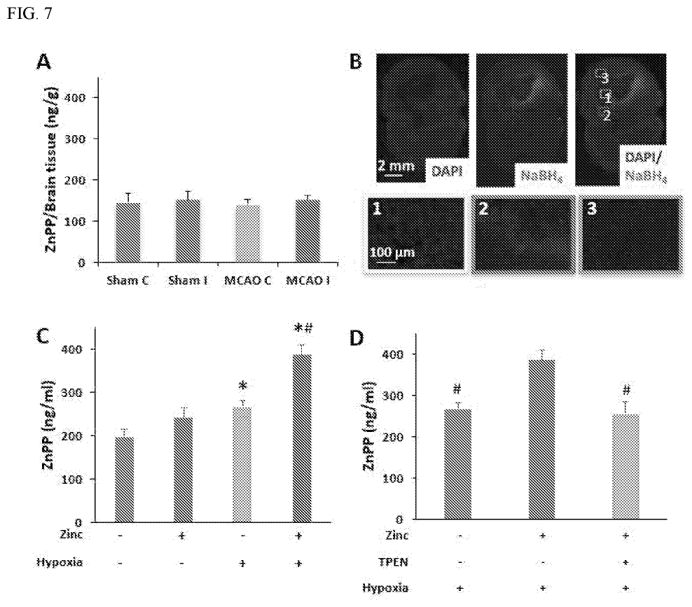

Normally, a limited amount of ZnPP exists in the brain. However, the data in FIGS. 3-5 show higher levels of ZnPP in hemorrhagic brain following ICH. To investigate whether hypoxia is also present at the ICH edema region following ICH, brain oxygen levels in the hemorrhagic rats were measured by in vivo EPR oximetry. The brain partial pressure of oxygen (pO₂) level at the edge of hemorrhagic area, where the crystal was implanted (FIG. 6, arrowed) and where high concentrations of ZnPP were detected, was decreased by about one third, from 33.4±3.1 mmHg before collagenase injection to 22.7±1.7 mmHg at 24 hours after collagenase injection and ICH induction (P=0.017) (FIG. 6). Since the brain is known to upregulate erythropoietin expression (and thus heme biosynthesis) by hypoxia, lowered pO₂ could drive heme synthesis, and thus protoporphyrin and ferrochelatase levels.

The two factors identified in ZnPP formation, hypoxia and excess free zinc, are also present in ischemic stroke where there is brain hypoxia without any blood release into brain parenchyma. An MCAO rat model of ischemic stroke was used to evaluate whether blood is required for ZnPP formation. FIG. 7A shows minimal ZnPP in ischemic brain tissue, indicating that ZnPP formation is in some manner dependent upon blood in the brain parenchyma. Insignificant ZnPP levels at the center of hemorrhagic area (FIG. 3). NaBH₄ was used to visualize the hemorrhage and DAPI was used to stain the cells. As shown in FIG. 7B, the center of the hemorrhagic area, where ZnPP was not detectable, has abundant blood but only a few cell (FIG. 7B, area 1). ZnPP also was hard to detect at the area where brain hemorrhage was invisible in brain tissue (FIG. 7B, area 3). A high level ZnPP was detected in the brain parenchyma (FIG. 7B, area 2), where excess free zinc, hypoxia and blood are all present.

Although most cells in blood are enucleated mature erythrocytes that are incapable of heme biosynthesis, a small portion of blood cells are nucleated (e.g., white cells) and are capable of heme biosynthesis. Therefore, the ability of whole blood to generate appreciable ZnPP was evaluated. 100 μM zinc was added to whole rat blood under normoxia or hypoxia, and increased ZnPP generation under hypoxia conditions was observed (P=0.0015), compared to normoxia (P=0.12) (FIG. 7C). Furthermore, as shown in FIG. 7D, treatment with TPEN decreased the zinc-induced ZnPP generation in hypoxic blood (P=0.00096).

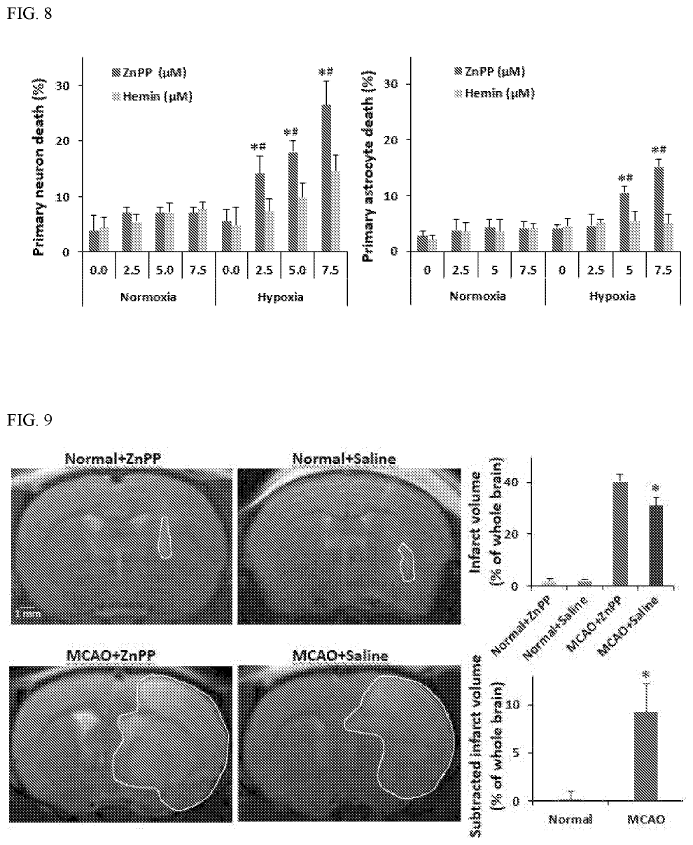

ZnPP is toxic to brain cells and tissue under hypoxia but not under normoxia. FIG. 8 shows that the toxicity of ZnPP in primary neurons and astrocytes is greater than at normoxia and it is significantly higher than the toxicity due to hemin, a recognized toxic factor in ICH. Neither ZnPP nor hemin induced significant cell death in normoxia at any concentration tested. However, under hypoxia conditions, both ZnPP and hemin showed significant dose-dependent increases under neuron toxicity (FIG. 8, left). ZnPP toxicity in hypoxia was greater than that of hemin, (2.5 μM: P=0.0092; 5 μM: P=0.0046; 7.5 μM: P=0.0059) which has long been recognized as a major cause of brain damage following ICH. Analogous experiments with primary astrocytes exhibited similar results (FIG. 8, right), but only ZnPP showed a dose dependent increase in toxicity in hypoxia while hemin did not (5 μM ZnPP: P=0.0071, hypoxia vs. normoxia, P=0.039 vs. hemin; 7.5 μM ZnPP: P=0.0084, hypoxia vs. normoxia, P=0.0012 vs. hemin). These results demonstrate that ZnPP is toxic to both neuron and astrocytes, especially at hypoxia.

To further investigate ZnPP toxicity at hypoxia in vivo, ZnPP was injected to the brain parenchyma of the ischemic side (right) of permanent middle cerebral artery occlusion (MCAO) rat model. The MRI T2-weighted images (FIG. 9) showed that the brain damage area is significantly increased by ZnPP injection in MCAO rats. The increase of brain damage only in MCAO rats but not in normal rats indicates that ZnPP is toxic only at hypoxia. Since hypoxia occurs following hemorrhagic stroke, brain damage associated with hemorrhagic stroke involves ZnPP.

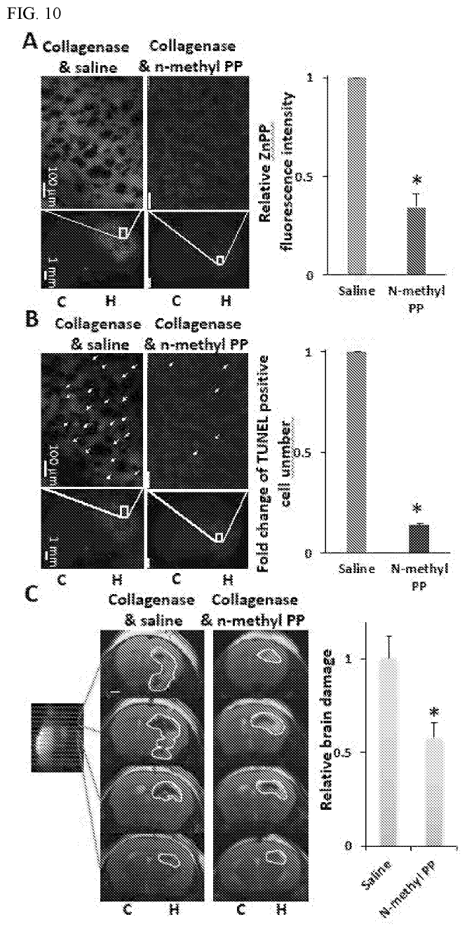

Ferrochelatase inhibition decreased ZnPP generation and brain damage after ICH. The final reaction in heme biosynthesis is iron insertion into protoporphyrin IX by mitochondrial ferrochelatase. Ferrochelatase can be potently inhibited by N-methyl protoporphyrin IX (N-methyl PP). N-methyl PP was injected intraperitoneally one hour before ICH induction. As shown in FIG. 10A, N-methyl PP treatment reduced the ZnPP fluorescence in brain tissue by about two thirds of that without N-methyl PP treatment (P=0.0037), showing that administering a ferrochelatase inhibitor decreased ZnPP generation after ICH. It also indicates that the ICH-induced ZnPP generation is caused by ferrochelatase inserting zinc into protoporphyrin. At the same time, brain cell death was measured by TUNEL to investigate the toxicity of ZnPP in ICH. As shown in FIG. 10B, the number of TUNEL positive cells with N-methyl PP pretreatment decreased to just 14% of those in saline treated control group. This significant reduction of cell death from N-methyl PP (P=0.000039) indicates that ZnPP is involved in ICH-induced brain damage. To further confirm the inhibition of ferrochelatase could be effective in decreasing brain tissue damage in ICH, rats were pretreated with N-methyl PP one hour before collagenase-induced ICH, and brain damage was detected by T2-weighted MRI. FIG. 10C shows that N-methyl PP pre-treatment significantly reduced the brain damage in ICH (P=0.043), indicating endogenous ZnPP promotes brain damage following ICH.

This disclosure provides the first evidence that zinc is accumulated following ICH, which leads to brain damage around the hemorrhagic area following ICH. The accumulated zinc combines with protoporphyrin IX to form endogenous ZnPP following ICH. Moreover, hypoxia occurs around hemorrhagic areas and the in vitro and in vivo results described herein suggest that ZnPP is toxic to brain under hypoxic condition. In conclusion, the data resented in this disclosure demonstrates that ICH triggers zinc accumulation around hemorrhagic area, which contributes to the ICH-induced brain damage via ZnPP generation. The findings provide a novel mechanism accounting for intracerebral hemorrhage-induced brain injury and it also suggests that zinc is a new potential target for reducing brain damage following intracerebral hemorrhage.

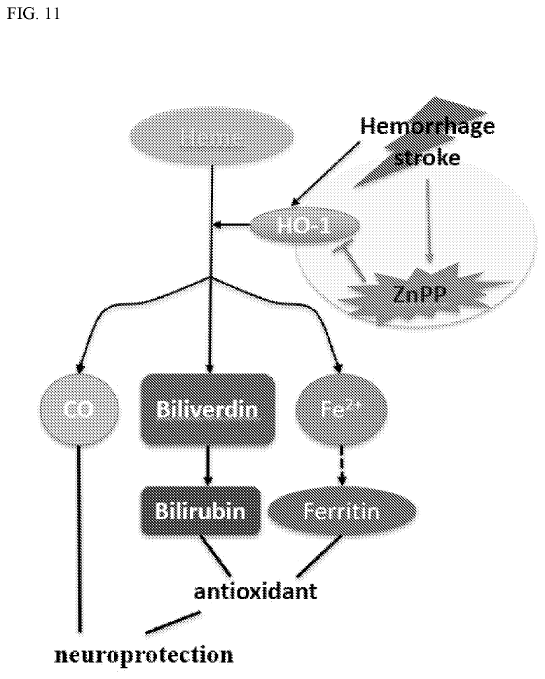

Without wishing to be bound by any particular theory or mechanism of action, ZnPP is a well-known potent inhibitor of heme oxygenase-1 (HO-1) with levels as low as 0.15 significantly decreasing HO-1 activity and with 50% inhibition at about 2.5 μM. HO-1 is known to be neuroprotective, participating in heme breakdown (FIG. 11). Heme is neurotoxic through accelerating the generation of reactive oxygen species (ROS). Thus, endogenous formation of ZnPP may block the breakdown of heme by inhibiting HO-1, thereby increasing the amount of heme available to damage brain tissue. One product from the reaction of heme and HO-1, carbon monoxide (CO), is a neuroprotective agent in hemorrhagic shock. A downstream product from the reaction of heme and HO-1, bilirubin, is a strong antioxidant. Since neuroprotection is provided by these HO-1 products, ZnPP inhibition of HO-1 may further promote brain damage. In summary, currently, it is known that heme and its products are neuroprotective following ICH (FIG. 11). However, an endogenous factor, ZnPP, may interfere with the formation of neuroprotective factors. Reducing ZnPP levels can reduce the extent to which ZnPP can interfere with the generation of neuroprotective factors. Accordingly, reducing ZnPP levels can reduce damage to brain tissue resulting from ICH.

Thus, this disclosure describes compositions and methods for treating hemorrhagic stroke. As used herein, “treat” or variations thereof refer to reducing, limiting progression, ameliorating, or resolving, to any extent, the symptoms or signs related to hemorrhagic stroke. A “treatment” may be therapeutic or prophylactic. “Therapeutic” and variations thereof refer to a treatment that ameliorates one or more existing symptoms or clinical signs associated with hemorrhagic stroke. “Prophylactic” and variations thereof refer to a treatment that limits, to any extent, the development and/or appearance of a symptom or clinical sign of hemorrhagic stroke. Generally, a “therapeutic” treatment is initiated after hemorrhagic stroke manifests in a subject, while “prophylactic” treatment is initiated before hemorrhagic stroke manifests in a subject.

Generally, the composition includes an inhibitor of ferrochelatase in an amount effective to decrease the extent to which protoporphyrin IX combines with zinc to form zinc protoporphyrin (ZnPP). While described herein in the context of an exemplary embodiment in which the ferrochelatase inhibitor is N-methyl protoporphyrin IX, the composition can include, and the methods may be practiced using any suitable ferrochelatase inhibitor. Exemplary alternative ferrochelatase inhibitors include alternative protoporphyrin isomers. Porphyrins have many isomers depending upon the ordering of substituents around the outer portion of the ring. Human protoporphyrin IX is the free base ligand of heme, but there are 15 isomers of protoporphyrin, each of which includes four methyl groups, two vinyl groups, and two propionic groups. Thus, alternative isomers of protoporphyrin may inhibit ferrochelatase. Additional alternative ferrochelatase inhibitors include modifications of the protoporphyrin, whether protoporphyrin IX or another isomer of protoporphyrin. Exemplary modifications include reducing the vinyl groups to alkyl groups (e.g., mesoporphyrins) and/or different N-alkylations. Exemplary suitable N-alkylations of protoporphyrins, whether protoporphyrin IX or another isomer and/or whether a protoporphyrin or a mesoporphyrin, can include an alkyl chain of one to four carbons such as, for example, methyl, ethyl, propyl, n-propyl, isopropyl, t-butyl, n-butyl, sec-butyl, or isobutyl. Additional exemplary ferrochelatase inhibitors include protein kinase inhibitors such as, for example, axitinib, cabozantinib, crenolanib, erlotinib, gefitinib, linsitinib, momelotinib, neratinib, nilotinib, pelitinib, vargatef, vemurafenib, AEW-541, ARRY-380, AZD-2014, AZD-5438, AZD-8055, BGT-226, BMS-690514, CP-724714, CUDC-101, Cyc-116, E-7080, GSK-1070916, GSK-690693, MK-2461, MK-8033, OSI-027, and/or R-406.

Treating hemorrhagic stroke can be prophylactic or, alternatively, can be initiated after the subject exhibits one or more symptoms or clinical signs of hemorrhagic stroke. Treatment that is prophylactic—e.g., initiated before a subject manifests a symptom or clinical sign of hemorrhagic stroke such as, for example, partial or total loss of consciousness; vomiting or sever nausea combined with another symptom; sudden numbness or weakness in the face, arm, or leg, especially on one side of the body; and/or sudden severe headache with no known cause—is referred to herein as treatment of a subject that is “at risk” of having hemorrhagic stroke. As used herein, the term “at risk” refers to a subject that may or may not actually possess the described risk. Thus, for example, a subject “at risk” of a hemorrhagic stroke is a subject possessing one or more risk factors associated with hemorrhagic stroke such as, for example, age, sex, ancestry, family history, a history of high blood pressure (e.g., consistently more than 120/80), excessive use of alcohol or drugs, smoking, using anti-blood clotting medication, presence of a biomarker indication disruption of the blood-brain barrier (e.g., the biomarker described in U.S. Patent Application Publication No. 2014/0314737 A1 or U.S. Patent Application Publication No. 2015/0198617 A1), and/or having any type of blood clotting disorder (e.g., hemophilia or sickle cell anemia).

Accordingly, a composition can be administered before, during, or after the subject first exhibits a symptom or clinical sign of hemorrhagic stroke. Treatment initiated before the subject first exhibits a symptom or clinical sign associated with hemorrhagic stroke may result in decreasing the likelihood that the subject experiences clinical evidence of hemorrhagic stroke compared to a subject to whom the composition is not administered and/or decreasing the severity of symptoms and/or clinical signs of hemorrhagic stroke. Treatment initiated after the subject first exhibits a symptom or clinical sign associated with hemorrhagic stroke may result in decreasing the severity of symptoms and/or clinical signs of hemorrhagic stroke compared to a subject to whom the composition is not administered.

Thus, the method includes administering an effective amount of the composition to a subject having, or at risk of, hemorrhagic stroke. In this aspect, an “effective amount” is an amount effective to reduce, limit progression, or ameliorate, to any extent, a symptom or clinical sign related to hemorrhagic stroke.

A ferrochelatase inhibitor may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the ferrochelatase inhibitor, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the ferrochelatase inhibitor without causing any undesirable biological effects or interacting in a deleterious manner with any of the other components of the pharmaceutical composition in which it is contained.

The ferrochelatase inhibitor may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, intrathecal etc.), or topical (e.g., intranasal, intrapulmonary, intradermal, transcutaneous, etc.). A composition also can be administered via a sustained or delayed release.

Thus, the pharmaceutical composition may be provided in any suitable form including but not limited to a solution, a suspension, an emulsion, a spray, an aerosol, or any form of mixture. The composition may be delivered in formulation with any pharmaceutically acceptable excipient, carrier, or vehicle.

A formulation may be conveniently presented in unit dosage form and may be prepared by methods well known in the art of pharmacy. Methods of preparing a composition with a pharmaceutically acceptable carrier include the step of bringing the ferrochelatase inhibitor into association with a carrier that constitutes one or more accessory ingredients. In general, a formulation may be prepared by uniformly and/or intimately bringing the ferrochelatase inhibitor into association with a liquid carrier, a finely divided solid carrier, or both, and then, if necessary, shaping the product into the desired formulations.

The amount of ferrochelatase inhibitor administered can vary depending on various factors including, but not limited to, the specific ferrochelatase inhibitor being administered, the weight, physical condition, and/or age of the subject, and/or the route of administration. Thus, the absolute weight of ferrochelatase inhibitor included in a given unit dosage form can vary widely, and depends upon factors such as the species, age, weight and physical condition of the subject, and/or the method of administration. Accordingly, it is not practical to set forth generally the amount that constitutes an effective amount of ferrochelatase inhibitor effective for all possible applications. Those of ordinary skill in the art, however, can readily determine the appropriate amount with due consideration of such factors.

For example, certain ferrochelatase inhibitors may be administered at the same dose and frequency for which the ferrochelatase inhibitor has received regulatory approval. In other cases, certain ferrochelatase inhibitor may be administered at the same dose and frequency at which the drug is being evaluated in clinical or preclinical studies. One can alter the dosages and/or frequency as needed to achieve a desired level of ferrochelatase inhibitor. Thus, one can use standard/known dosing regimens and/or customize dosing as needed. In some embodiments, the method can include administering sufficient ferrochelatase inhibitor to provide a dose of, for example, from about 100 ng/kg to about 100 mg/kg to the subject, although in some embodiments the methods may be performed by administering ferrochelatase inhibitor in a dose outside this range.

Thus, the ferrochelatase inhibitor may be administered to provide a minimum dose of at least 100 ng/kg, such as, for example, at least 500 ng/kg, at least 1 μm/kg, at least 5 μg/kg, at least 10 μg/kg, at least 20 μg/kg, at least 30 μg/kg, at least 40 μg/kg, at least 50 μg/kg, at least 60 μg/kg, at least 70 μg/kg, at least 80 μg/kg, at least 90 μg/kg, at least 100 μg/kg, at least 200 μg/kg, at least 250 μg/kg, at least 500 μg/kg, at least 750 μg/kg, at least 1 mg/kg, at least 5 mg/kg, at least 10 mg/kg, at least 15 mg/kg, at least 20 mg/kg, or at least 25 mg/kg.

The ferrochelatase inhibitor may be administered to provide a maximum dose of no more than 100 mg/kg, such as, for example, no more than 90 mg/kg, no more than 75 mg/kg, no more than 50 mg/kg, no more than 40 mg/kg, no more than 30 mg/kg, no more than 20 mg/kg, no more than 10 mg/kg, no more than 5 mg/kg, no more than 4 mg/kg, no more than 3 mg/kg, no more than 2 mg/kg, no more than 1 mg/kg, no more than 750 μg/kg, no more than 500 μg/kg, no more than 400 μg/kg, no more than 300 μg/kg, no more than 200 μg/kg, no more than 100 μg/kg, no more than 50 μg/kg, no more than 25 μg/kg, no more than 10 μg/kg, no more than 5 μg/kg, or no more than 1 μg/kg.

In some embodiments, the ferrochelatase inhibitor may be administered to provide a dose characterized by any range that includes, as endpoints, any combination of a minimum dose identified above and any maximum dose identified above that is greater than the minimum dose. For example, in some embodiments, the ferrochelatase inhibitor may be administered to provide a dose of from 10 μg/kg to about 100 mg/kg to the subject, for example, a dose of from about 30 μg/kg to about 20 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, the method can include administering sufficient ferrochelatase inhibitor to provide a dose of, for example, from about 0.01 mg/m² to about 10 mg/m².

In some embodiments, ferrochelatase inhibitor may be administered, for example, from a single dose to multiple doses per week, although in some embodiments the method can be performed by administering ferrochelatase inhibitor at a frequency outside this range. In certain embodiments, a ferrochelatase inhibitor may be administered from a single administration to multiple times day. In certain embodiments, the ferrochelatase inhibitor may be administered once per day, twice per day, or three times per day. In other embodiments, the ferrochelatase inhibitor may be administered on an “as needed” basis. As used herein, “as needed” refers to a dosing regimen that may be inconsistent between specified periods, but does not exceed the maximum frequency deemed safe for the dose being administered. Accordingly, the maximum frequency depends at least in part on the dosage being administered. Those of skill in the art can readily determine the maximum frequency that a given dose may be administered to a particular subject.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Rat Model of Intracerebral Hemorrhage

The Laboratory Animal Care and Use Committee of the University of New Mexico approved all experimental protocols. The animals were used in compliance with the NIH Guide for Care and Use of Laboratory Animals. Male Sprague Dawley rats (Charles River Laboratories, Inc., Wilmington, Mass.) weighing 290 g to 320 g were anesthetized with isoflurane (5% for induction, 2% for maintenance) in N₂O/O₂ (70:30%) during surgical procedures, and the body temperature was maintained at 37.5° C.±0.5° C. using a heating pad. A small burr hole was made at 3.5 mm right of the bregma. 0.2 U collagenase (Sigma-Aldrich, St. Louis, Mo.) in 1 μl sterile saline was injected to 6.0 mm deep through the burr hole. The needle was removed at five minutes after collagenase injection to prevent backflow. A total of 49 rats were used in this study.

### Mouse Model of Intracerebral Hemorrhage

The Laboratory Animal Care and Use Committee of the University of New Mexico approved all experimental protocols. The animals were used in compliance with the NIH Guide for Care and Use of Laboratory Animals. Male C57BL/6 (Charles River Laboratories, Inc., Wilmington, Mass.) weighing 16 g to 20 g were anesthetized with isoflurane (5% for induction, 1.5% for maintenance) in N₂O/O₂ (70:30%) during surgical procedures, and the body temperature was maintained at 37.5° C.±0.5° C. using a heating pad. A small burr hole was made at 2 mm right of the bregma. 0.075 U collagenase (Sigma-Aldrich, St. Louis, Mo.) in 0.5 μl sterile saline was injected to 3.7 mm deep through the burr hole. The needle was removed at 10 minutes after collagenase injection to prevent backflow. A total of ten mice were used in this study.

### Magnetic Resonance Imaging

All multimodal magnetic resonance imaging (MM) of the rat was performed by using a 4.7-Tesla MM scanner (Bruker Biospin, Billerica, Mass.), which was equipped with a 40-cm bore, a 660 mT/m (rise time within 120 μs) gradient and shim systems (Bruker Biospin, Billerica, Mass.). The rats were anaesthetized with 2% isoflurane (Clipper Distributing Company, LLC, St. Joseph, Mo.) during MM. At the same time respiration and heart rate were monitored. The body temperature of the rats was maintained at 37.0° C.±0.5° C. The success of the intracerebral hemorrhage model and brain damage was estimated by T2-weighted images. T2-weighted images were acquired with a fast spin-echo sequence (rapid acquisition with relaxation enhancement (RARE)) (Repetition Time (TR)/Echo Time (TE)=5,000 ms/56 ms, Field of View (FOV)=4 cm×4 cm, slice thickness=1 mm, inter-slice distance=1.1 mm, number of slices=12, matrix=256×256, number of average=3).

### TPEN Administration

N,N,N′,N′-tetrakis-(2-Pyridylmethyl)ethylenediamine (TPEN, Sigma-Aldrich, St. Louis, Mo.) was dissolved in dimethyl sulfoxide (DMSO) to 25 mg/ml and then further diluted in physiological saline to a final concentration of 2.5 mg/ml. 10 mg/kg TPEN was intraperitoneal injected at one hour before collagenase injection. Saline with 10% DMSO was used as control.

### N-Methyl Protoporphyrin IX Administration

N-methyl protoporphyrin IX (Santa Cruz Biotechnology, Inc., Dallas, Tex.), a ferrochelatase inhibitor was dissolved in DMSO to 200 mg/ml and then further diluted in physiological saline to a final concentration of 20 mg/ml. 100 mg/kg N-methyl protoporphyrin IX was intraperitoneally injected one hour before collagenase injection. Saline with 10% DMSO was used as control.

### Rat Model of Focal Cerebral Ischemia and ZnPP Treatment

Middle cerebral artery occlusion (MCAO) surgery was used to induce focal cerebral ischemia through intraluminal filament methods as described previously (Zhao et al., 2014, Stroke 45:1139-1147). The animals underwent right MCAO for 24 hours. ZnPP (10 μg/kg) in 5 μl saline or 5 μl saline was injected to 6.0 mm deep at 3.5 mm right of the bregma through a burr hole on normal or MCAO rat, 10 minutes after MCAO onset.

### Tissue Processing

At 24 hours after collagenase injection, the rats were transcardially perfused with cold PBS and then 4% paraformaldehyde. After that, the brain was taken out and fixed in 4% paraformaldehyde. The brain was placed in 20% sucrose after fixed. The brain was embedded in OCT solution for cryosectioning after it sank to the bottom of the sucrose. 20-μm-think brain cryosections were prepared for zinc and cell death measurement.

### Staining for Labile Zinc

To detect labile zinc in brain tissue, cryosections 16 μm thick were stained with the zinc-specific membrane-permeable fluorescent dyes Fluozin-3 (Invitrogen, Thermo Fisher Scientific, Carlsbad, Calif.). The cryosections were washed in saline and incubated with Fluozin-3 (5 μM) for 15 minutes at room temperature. After washing in saline, images were acquired by a fluorescence microscope (Olympus IX71, Olympus Corp., Center Valley, Pa.) with a GFP dichroic mirror.

### Examination of Brain Cell Death

A standard terminal deoxynucleotidyl transferase-mediated dUTP nick-end labeling (TUNEL) procedure for frozen tissue sections was performed (Click-iT TUNEL Alexa Fluor 488 Imaging Assay kit (Thermo Fisher Scientific, Waltham, Mass.). Histological images were captured on a fluorescence microscope (Olympus IX71, Olympus Corp., Center Valley, Pa.) with a TRITC dichroic mirror.

### Detection of ZnPP by Liquid Chromatography Mass Spectroscopy (LCMS)

Brain tissue was harvested at 24 hours after collagenase injection. The hemorrhagic hemisphere was homogenized on ice in ethanol, after centrifugation at 13,000×g, the supernatant was analyzed for ZnPP using C-18 reverse-phase HPLC column. The right hemisphere of normal rat was used as control. The elution was carried out at a flow rate of 2 ml/min. Mass spectra of ZnPP were recorded in 300-700 range. Identification of ZnPP was made on the basis of its matching peak with standard ZnPP.

### Detection of ZnPP in Brain Tissue by Supercritical Fluid Chromatography-Mass Spectrometry (SFC-MS)

Brain tissue was harvested at 24 hours after collagenase injection. The hemorrhagic hemisphere was homogenized on ice in ethanol, after centrifugation at 13,000×g, SFC-MS analysis was performed on the supernatant using with Acquity UPC2 Ultra-Performance Liquid Chromatograph coupled with a single quadrupole-mass detector (SQD) (Waters Corporation, Milford, Mass.). The LC-MS system was controlled by MassLynx Version 4.1 software. SFC condition: Acquity UPC2 HSS C18 column (2.1×100 mm, 1.8 μm) at 40° C. Mobile phase A: CO₂ in liquid state. Mobile phase B: methanol. Gradient: 0.00 min: A/B (100/0); 2.00 min: A/B (75/25); 5.90 min: A/B (75/25); 5.91 min: A/B (100/0). Flow rate was at 2.00 mL/min of the mobile phase were set at 1.75 mL/min. The right hemisphere of a saline injected rat brain was used as control. ZnPP were detected in positive electrospray ionization mode (Est). The ion source temperature was 150° C. N₂ was used as the desolvation gas at a flow rate of 500 L/h at 350° C. Voltages of the capillary and the cone were 3 kV and 45 V, respectively. MS detection was in the m/z range of 300 to 700. The ZnPP protonated ion was observed at m/z of 626.0. Identification of ZnPP was made on the basis of its matching peak with standard ZnPP.

### Fluorescence Analysis of ZnPP

The animals were euthanized at 24 hours after collagenase injection. The brains were harvested and cut into 2-mm thick coronal slice. The tissue with visible blood at the hemorrhagic hemisphere of each slice was collected. And the tissue at the corresponding location at the contralateral hemisphere was also collected. The tissue was homogenized on ice in ethanol. After centrifugation at 13,000×g, the fluorescence intensity (excitation=416 nm, emission=588 nm) was determined using a SpectraMax M2 Multi-Mode Microplate Readers (Molecular Devices LLC, Sunnyvale, Calif.) as previously described (Nakamura et al., 2011. J Control Release 155(3):367-375. After being treated with heparin as an anti-coagulant, 50 μl blood was diluted in ethanol. Followed by centrifugation, the fluorescence intensity of ZnPP in the ethanol extract supernatant was measured.

### Hemorrhagic Measurement by Fluorescence Microscopy

To visualize the hemorrhage in brain tissue, cryosections (16-μm-thick) were treated with 0.2% (W/V) NaBH₄ in PBS (Liu et al., 2002, J Cerebral Blood Flow Metab 22:1222-1230) for 15 minutes. After a five-minute rinsing in PBS, the section was mounted in PROLONG Gold Antifade Mountant with DAPI (Thermo Fisher Scientific, Waltham, Mass.). Images were acquired by a fluorescence microscope (Olympus IX71) with TRICT (Hemorrhage, NaBH4) and DAPI (nucleus) dichroic mirrors.

### Measurement of Cerebral pO₂ by Electron Paramagnetic Resonance

Under anesthesia, a pin hole on the parietal skull was made at 4.5 mm right of the bregma. A LiPc crystal (approximate diameter 0.2 mm) was placed at a depth of 6 mm using a microdialysis guide cannula with an inner diameter of 0.24 mm (CMA Microdialysis AB, Kista, Sweden). The rats were allowed to recover from implantation 72 hours before electron paramagnetic resonance (EPR) measurement. Correct assignment of the implantation site was confirmed by MRI.

For measurement of local cerebral pO₂ in the anesthetized rats before and 24 hours after injection of collagenase, EPR oximetry was conducted according to previously described methods (Liu et al., 1993. Proc Natl Acad Sci USA 90(12):5438-5342; Liu et al., 1995. Brain Res 685(1-2):91-98; Shen et al., 2009. J Cereb Blood Flow Metab 29(10):1695-1703; Weaver et al., 2014. Toxicol Appl Pharmacol 275(2):73-78) using a Bruker EleXsys E540 EPR spectrometer equipped with an L-band bridge (Bruker Instruments, Billerica, Mass.).

### Exposure of Blood to Hypoxia Treatment

Blood was drawn from normal Sprague Dawley rats. Blood was subjected to hypoxia by incubating in a humidified airtight chamber (Billups-Rothenberg, Inc., San Diego, Calif.) equipped with an air lock and flushed with 95% N₂/5% CO₂ for 15 minutes. Then the chamber was scaled and kept at 37° C. for three hours.

### Cytotoxicity Assay

Astrocytic death rate was measured using Cytotox 96 nonradioactive cytotoxicity assay kit (Promega, Madison, Wis.), which quantitatively measures lactate dehydrogenase (LDH) release from dead cells. Astroytes (5×10³ cells/well) were seeded into 96-well microtiter plates. Following three hours normoxia or hypoxia treatment, 50 μl of the reconstituted substrate mixture was added to each well of the plate. Thirty minutes later, 50 μl of the stop solution was added to each well, and the absorbance was measured at 490 nm in a microplate reader (Bio-Rad 3350, Bio-Rad Laboratories, Inc. Hercules, Calif.). Triton-X 100-treated cells were used as 100% cell death control. The cell death rate was calculated by using the formula: Cell death rate=(Experimental absorbance value—culture medium absorbance value)/(Triton-X 100-treated absorbance value—culture medium absorbance value).

### Statistical Analysis

Data were presented as mean±SEM. The Student's t-test was used to analyze the differences in means from groups. A value of P<0.05 was considered statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A pharmaceutical composition comprising:
a ferrochelatase inhibitor; and
a pharmaceutically acceptable carrier.

2. The pharmaceutical composition of claim 1 wherein the ferrochelatase inhibitor comprises:
an isomer of a protoporphyrin compound; or
a chemically modified protoporphyrin analog.

3. The pharmaceutical composition of claim 2 wherein the protoporphyrin analog comprises:
reduction of at least one protoporphyrin vinyl group; or
N-alkylation.

4. The pharmaceutical composition of claim 3 wherein the N-alkylation comprises N-methylation.

5. The pharmaceutical composition of claim 1, wherein the ferrochelatase inhibitor comprises N-methyl protoporphyrin IX.

6. The pharmaceutical composition of claim 1 wherein the ferrochelatase inhibitor comprises a protein kinase inhibitor.

7. The pharmaceutical composition of claim 1 wherein the protein kinase inhibitor comprises axitinib, cabozantinib, crenolanib, erlotinib, gefitinib, linsitinib, momelotinib, neratinib, nilotinib, pelitinib, vargatef, vemurafenib, AEW-541, ARRY-380, AZD-2014, AZD-5438, AZD-8055, BGT-226, BMS-690514, CP-724714, CUDC-101, Cyc-116, E-7080, GSK-1070916, GSK-690693, MK-2461, MK-8033, OSI-027, or R-406.

8. A method of treating a subject having, or at risk of having, a hemorrhagic stroke, the method comprising:
administering to the subject an amount of the pharmaceutical composition of claim 1 effective to ameliorate at least one symptom or clinical sign of hemorrhagic stroke.

9. The method of claim 8 wherein the pharmaceutical composition is administered to the subject before the subject manifests a symptom or clinical sign of hemorrhagic stroke.

10. The method of claim 8 wherein the pharmaceutical composition is administered to the subject after the subject manifests a symptom or clinical sign of hemorrhagic stroke.

11. The method of claim 8 wherein the amount of the pharmaceutical composition effective to ameliorate at least one symptom or clinical sign of hemorrhagic stroke is an amount effective to inhibit protoporphyrin IX from combining with Zn to form zinc protoporphyrin (ZnPP).
